(19) United States
(12) Patent Application Publication
(10) Pub. No.: US 2025/0270484 A1
(43) Pub. Date: Aug. 28, 2025

(54) METHOD OF ACCELERATING MALTING PROCESS

(71) Applicants: Yin Li, New Berlin, WI (US); Gustavo Hector Strasser, Milwaukee, WI (US); Junyong Zhang, Winnipeg (CA)

(72) Inventors: Yin Li, New Berlin, WI (US); Gustavo Hector Strasser, Milwaukee, WI (US); Junyong Zhang, Winnipeg (CA)

(73) Assignee: Malteurop North America Inc., West Allis, WI (US)

(21) Appl. No.: 19/058,875

(22) Filed: Feb. 20, 2025

Related U.S. Application Data
(60) Provisional application No. 63/556,707, filed on Feb. 22, 2024.

(51) Int. Cl. C12C 1/047; A23L 7/20; C12C 1/02; C12C 1/067

(52) U.S. Cl. CPC C12C 1/047; A23L 7/20; C12C 1/02; C12C 1/067

(57) ABSTRACT

Embodiments of the disclosure relate to a method of accelerating a malting process. In the method, a grain is steeped in water including β-glucanase to produce chitted malt. The chitted malt is germinated to produce green malt, and during germinating, an aqueous mixture including β-glucanase and Gibberellic acid is applied to the chitted malt. The green malt is then kilned to reduce the moisture content to produce a batch of malt. Advantageously, the finished batch of malt exhibits lower levels of deoxynivalenol contamination than conventional malts.

### CROSS-REFERENCE TO RELATED APPLICATION

This application claims the benefit of priority to U.S. Provisional Application No. 63/556,707, filed on Feb. 22, 2024, the entire contents of which are incorporated herein by reference thereto.

### FIELD OF THE INVENTION

This invention generally relates to malting of grains and, in particular, to a method of accelerating the malting process.

### BACKGROUND OF THE INVENTION

Malting is a process of partially germinating a cereal grain to modify the grain's nutritional profile. In particular, malting involves the development of enzymes that convert the grain's starches into sugars, which can be fermented. Thus, malted grains, or malts, are commonly used in brewing and distilling. A variety of grains can be malted, including barley, wheat, rye, oat, rice, corn, and sorghum. In general, the malting requires steeping of the grain in water, followed by germination, and then kilning. Steeping and germinating grains may take several days. Thus, to increase production, typically new steeping vats and germinating tanks have to be constructed to hold the grain. As will be discussed in the present disclosure, Applicant has recognized a need to improve production, including without resorting to large capital expenditures for new vats and tanks.

### BRIEF SUMMARY OF THE INVENTION

In a first aspect, embodiments of the present disclosure relate to a method of accelerating a malting process. In the method, a grain is steeped in water including β-glucanase to produce chitted malt. The chitted malt is germinated to produce green malt, and during germinating, an aqueous mixture including β-glucanase and Gibberellic acid is applied to the chitted malt. The green malt is then kilned to reduce the moisture content to produce a batch of malt.

A second aspect of the disclosure relates to the method according to the first aspect in which the water comprises β-glucanase in an amount in a range from 25 ppm to 100 ppm by weight of the grain.

A third aspect of the disclosure relates to the method according to the first aspect or the second aspect in which the water further comprises xylanase.

A fourth aspect of the disclosure relates to the method according to any of the first aspect to the third aspect in which steeping is performed for a time of less than 30 hours.

A fifth aspect of the disclosure relates to the method according to any of the first aspect to the fourth aspect in which steeping comprises a wet phase and a dry phase and in which the dry phase is performed for a time of 10 hours or less.

A sixth aspect of the disclosure relates to the method according to any of the first aspect to the fifth aspect in which the aqueous mixture further comprises xylanase.

A seventh aspect of the disclosure relates to the method according to any of the first aspect to the sixth aspect in which the aqueous mixture comprises Gibberellic acid in an amount in a range from 0.023 ppm to 1.82 ppm by weight of grain.

An eighth aspect of the disclosure relates to the method according to any of the first aspect to the seventh aspect in which the aqueous mixture comprises β-glucanase in an amount in a range from 25 ppm to 100 ppm by weight of grain.

A ninth aspect of the disclosure relates to the method according to any of the first aspect to the eighth aspect in which the chitted malt is germinated at a temperature in a range from 16° C. to 22° C.

A tenth aspect of the disclosure relates to the method according to any of the first aspect to the ninth aspect in which the chitted malt is germinated at a relative humidity in a range of 95% to 100%.

An eleventh aspect of the disclosure relates to the method according to any of the first aspect to the tenth aspect in which the chitted malt comprises a moisture content in a range from 40% to 50% by weight.

An twelfth aspect of the disclosure relates to the method according to any of the first aspect to the eleventh aspect in which the malt comprises a moisture content in a range from 3.0% by weight to 6.0% by weight.

A thirteenth aspect of the disclosure relates to the method according to any of the first aspect to the twelfth aspect in which kilning is performed at a temperature in the range of 40° C. to 90° C. for a time of 22 hours to 36 hours.

A fourteenth aspect of the disclosure relates to the method according to any of the first aspect to the thirteenth aspect in which a first wort prepared from a first portion of the batch of the malt comprises a first β-glucan level, in which a second wort prepared from a second portion of the batch of malt, after pearling to remove husks, comprises a second β-glucan level, and in which the second β-glucan level is within 30 ppm of the first β-glucan level.

A fifteenth aspect of the disclosure relates to the method according to any of the first aspect to the fourteenth aspect in which, during steeping and germinating, the β-glucanase penetrates the grain to accelerate the steps of steeping and germinating.

A sixteenth aspect of the disclosure relates to the method according to any of the first aspect to the fifteenth aspect in which, for a given time period and for a given equipment setup, the method produces more batches of malt than for a method in which β-glucanase is not used during steps of steeping and germinating.

A seventeenth aspect of the disclosure relates to the method according to any of the first aspect to the sixteenth aspect in which the grain comprises at least one of barley, wheat, oat, or rye.

An eighteenth aspect of the disclosure relates to the method of any of the first aspect to the seventeenth aspect in which, prior to the steeping, the grain comprises a first concentration of deoxynivalenol (DON), the batch of malt comprises a second concentration of DON, and the second concentration is 70% or less of the first concentration.

A nineteenth aspect of the disclosure relates to the method of eighteenth aspect in which the second concentration of DON is 2.0 ppm or less.

A twentieth aspect of the disclosure relates to the method of the eighteenth aspect or the nineteenth aspect in which the second concentration of DON is 1.5 ppm or less.

A twenty-first aspect of the disclosure relates to a malting process. In the malting process, a grain is steeped in water. The water comprises an enzyme component selected from a group consisting of β-glucanase, xylanase, and combinations thereof, and during steeping, the enzyme component starts an initial modification of the grain. The grain is germinated while applying water comprising Gibberellic acid and the enzyme component to the grain. The initial modification expedites movement of water into the grain by increasing the rate at which micropyles of the grain open. The Gibberellic acid and the enzyme component penetrate the grain at the onset of germinating to facilitate faster and more effective water absorption and enzymatic activity than applying water alone. An aleurone layer of each grain is stimulated to cause secretion of endogenous enzymes as the water comprising Gibberellic acid and the enzyme component continues to infiltrate the grain. The endogenous enzymes break down reserves in the grain to cause continued modification of the grain. Cell walls within the grain are degraded through the use of the enzyme component that has infiltrated the grain and the endogenous enzymes such that modification of the grain takes less time than through modification using the endogenous enzymes alone.

A twenty-second aspect of the disclosure relates to a batch of malted grain formed through accelerated malting of a grain. The grain has an initial concentration of deoxynivalenol (DON), and the batch of malted grain having a final concentration of DON. The final concentration of DON is 70% or less of the initial concentration of DON.

A twenty-third aspect of the disclosure relates to the batch of malted grain of the twenty-second aspect in which the final concentration of DON is 60% or less of the initial concentration of DON.

A twenty-fourth aspect of the disclosure relates to the batch of malted grain of the twenty-second aspect or the twenty-third aspect in which the final concentration of DON is 50% or less of the initial concentration of DON.

A twenty-fifth aspect of the disclosure relates to the batch of malted grain of any of the twenty-second aspect to the twenty-fourth aspect in which the final concentration of DON is 2.0 ppm or less.

A twenty-sixth aspect of the disclosure relates to the batch of malted grain of any of the twenty-second aspect to the twenty-fifth aspect in which the final concentration of DON is 1.5 ppm or less.

A twenty-seventh aspect of the disclosure relates to the batch of malted grain of any of the twenty-second aspect to the twenty-sixth aspect in which the final concentration of DON is 1.0 ppm or less.

A twenty-eighth aspect of the disclosure relates to the batch of malted grain of any of the twenty-second aspect to the twenty-seventh aspect in which the grain comprises at least one of barley, wheat, oat, or rye.

Other aspects, objectives and advantages of the invention will become more apparent from the following detailed description when taken in conjunction with the accompanying drawings.

### BRIEF DESCRIPTION OF THE DRAWINGS

The accompanying drawings incorporated in and forming a part of the specification illustrate several aspects of the present invention and, together with the description, serve to explain the principles of the invention. In the drawings:

FIG. 1 is a schematic process flow diagram of a method of accelerated malting, according to an exemplary embodiment;

FIG. 2 is a schematic representation of a barley grain which may undergo accelerated malting, according to an exemplary embodiment;

FIG. 3 is a graph of the β-glucan levels for a control malt and for a malt produced according to the disclosed accelerated malting process, according to an exemplary embodiment; and

FIGS. 4A-4E provide a diagram of important actions taken during the accelerated malting process with FIG. 4A providing a total overview of the accelerated malting process and FIGS. 4B-4E providing an overview of certain stages, according to exemplary embodiments.

While the invention will be described in connection with certain preferred embodiments, there is no intent to limit it to those embodiments. On the contrary, the intent is to cover all alternatives, modifications and equivalents as included within the spirit and scope of the invention as defined by the appended claims.

### DETAILED DESCRIPTION OF THE INVENTION

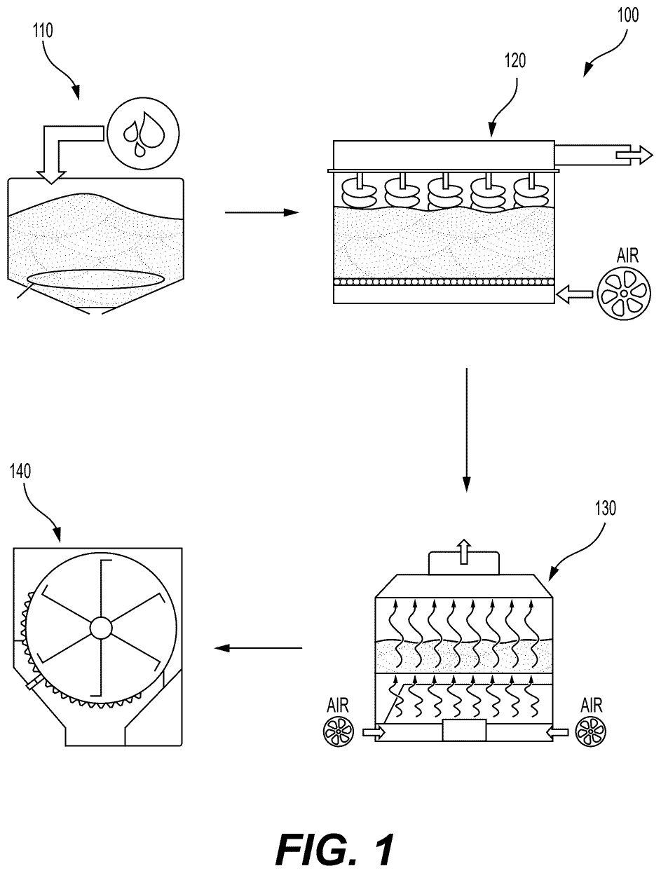

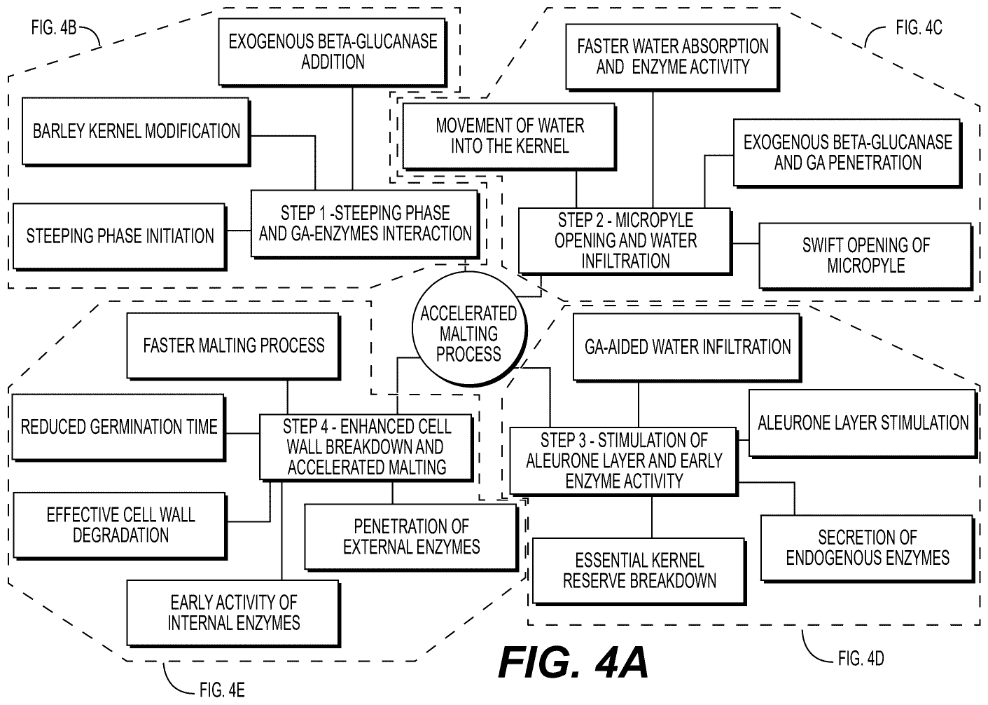

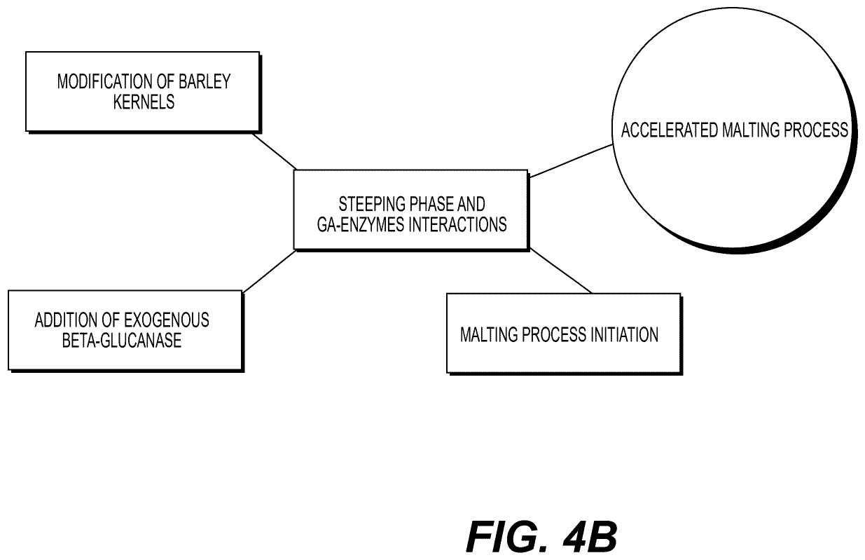

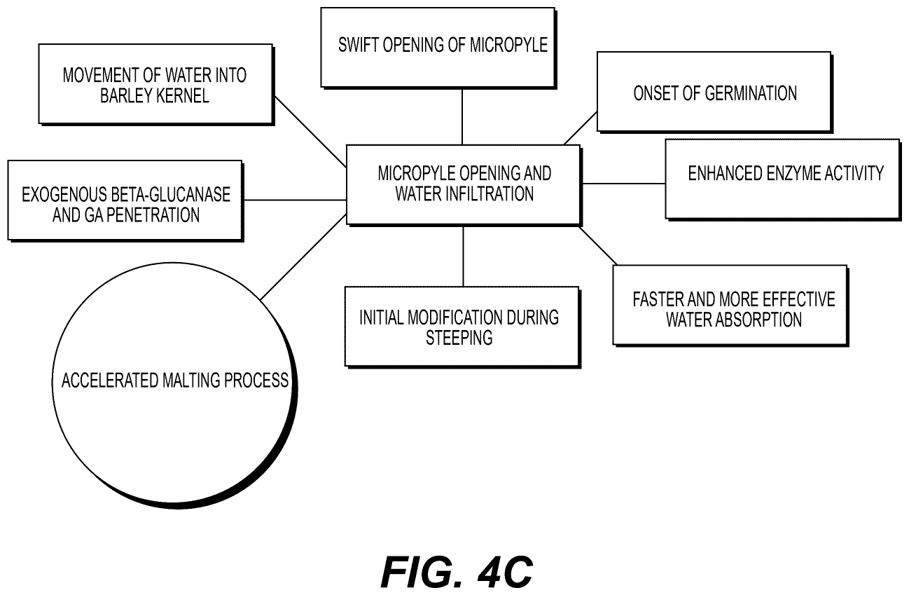

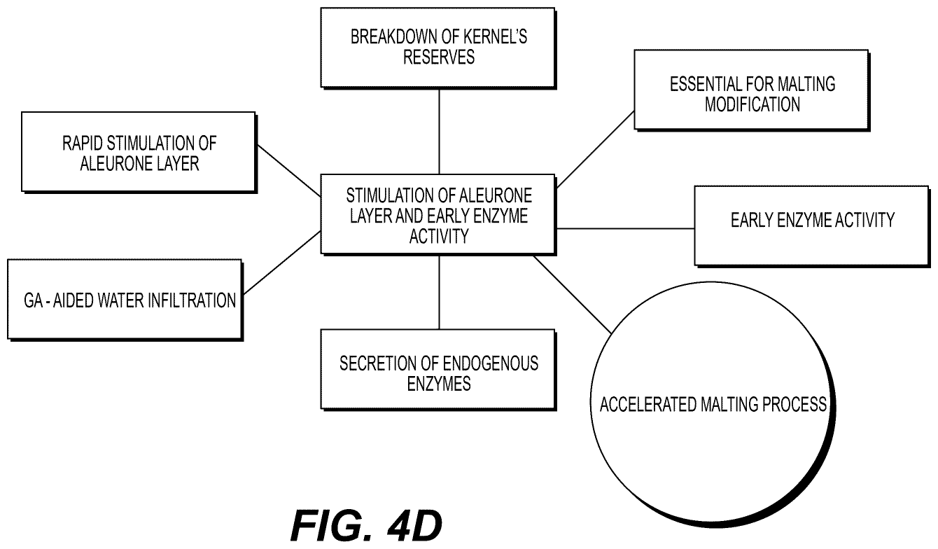

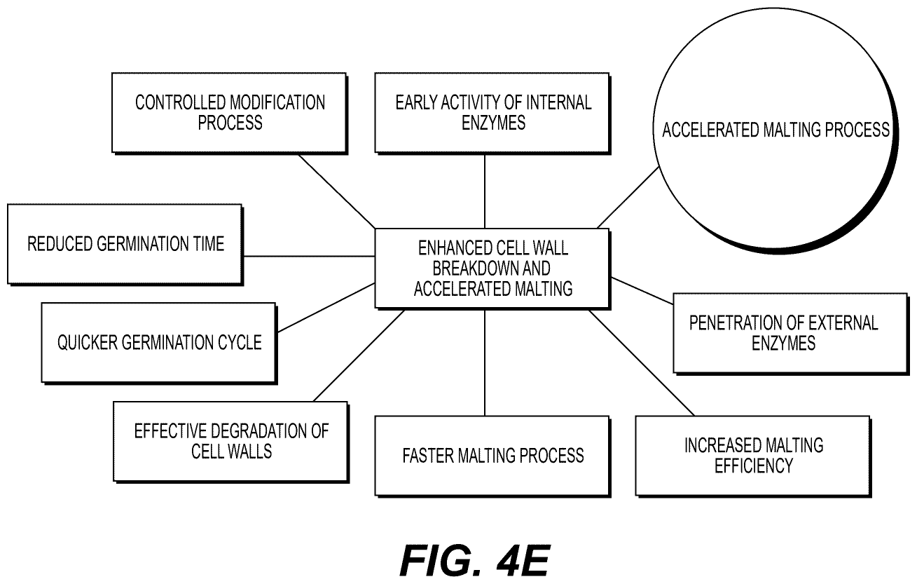

FIG. 1 provides a flow diagram of an accelerated malting process 100 for producing barley malt according to an exemplary embodiment. Additionally, FIGS. 4A-4E provide a diagram of important actions taken during the accelerated malting process with FIG. 4A providing a total overview of the accelerated malting process and FIGS. 4B-4E providing an overview of certain stages. The accelerated malting process 100 begins by steeping 110 a grain, such as barley, wheat, oat, or rye. During the step of steeping 110, the grain is soaked in a vat with water, which increases the moisture content of the barley. In one or more embodiments, the moisture content of the barley increases from, e.g., 10% to 15% by weight to 40% to 50% by weight. The steeping 110 is meant to stimulate the germination process of the barley. According to the present disclosure, during steeping 110, enzymes are added to accelerate the beginning of the germination process. In particular, β-glucanase or β-glucanase and xylanase are added in the water during steeping 110. In one or more embodiments, the water used during steeping 110 contains β-glucanase in an amount in a range from 25 ppm to 100 ppm by weight of raw grain (e.g., 25 kg to 100 kg of β-glucanase per 1 MT of grain). In one or more embodiments, the water during steeping 110 may additionally contain xylanase, and in such embodiments, the water includes the combination of β-glucanase and xylanase in an amount in a range from 25 ppm to 100 ppm by weight of raw grain. Further, in one or more embodiments, the β-glucanase has an enzyme activity of 35,000 U/g. In one or mor embodiments, the xylanase has an enzyme activity of 20,000 U/g. When used, the xylanase may help prevent high viscosity and filtration problems by reducing high molecular weight arabinoxylans content.

Steeping 110 may involve wet phase and dry phase over an extended period. During immersion, air is blown through the grain to provide dissolved oxygen, and the barley begins to germinate, which produces heat and carbon dioxide. During the dry phase, the water is removed to prevent excessive absorption, which could lead to rotting and uneven germination, and the carbon dioxide developed by the beginning of germination is removed from the grain. Conventionally, steeping 110 may take 30 to 40 hours or more. According to embodiments of the disclosed method, steeping 110 takes less than 30 hours. In one or more embodiments, steeping 110 is performed for 25 hours or less. In particular, the length of the air rests during steeping 110 may be substantially reduced. Conventionally, the air rests required more than 10 hours, in particular about 13 hours or more. According to embodiments of the disclosed method, the air rests during steeping 110 are performed for less than 10 hours, in particular for 5 hours or less.

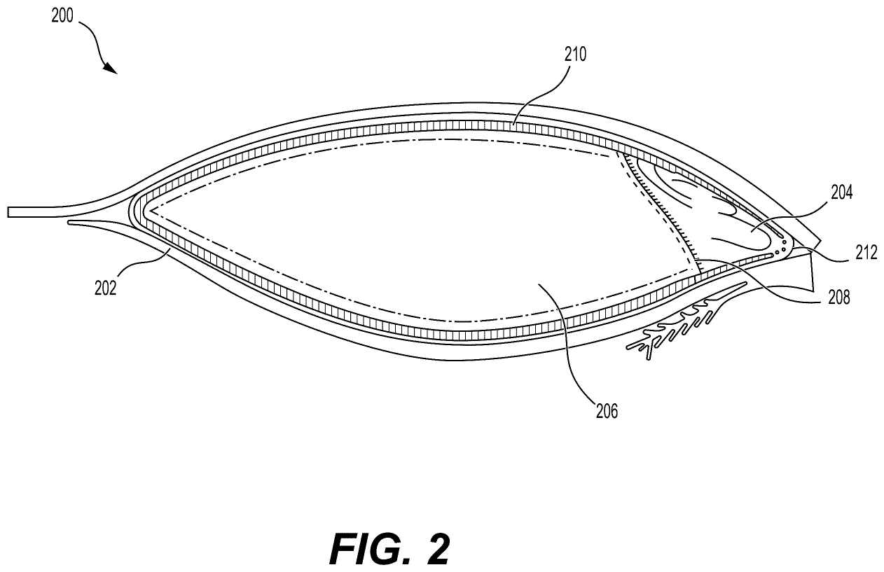

The time for steeping 110 is able to be reduced because the addition of enzymes during steeping 110 facilitates movement of water into the grains, which leads to a faster opening of the micropyle. FIG. 2 depicts a schematic illustration of a cross-section of a barley grain 200. While the discussion is framed in terms of a barley grain, the discussion is generally applicable to other grains, such as wheat, oat, and rye, among others. As can be seen in FIG. 2, the grain 200 includes a hull 202 surrounding an embryo 204 and a starchy endosperm 206. The embryo 204, if allowed to grow, would eventually turn into the roots and shoots of a barley plant. The endosperm 206 is the food reserve for the embryo 204 to fuel such growth. The embryo 204 and endosperm 206 are separated by a scutellum 208, and the endosperm is surrounded by an aleurone layer 210. During steeping, the embryo 204 is hydrated, and enzymes are released from the scutellum 208 and aleurone layer 210 of the grain 200 to breakdown the cell walls of the endosperm 206, releasing starch granules that are converted to sugars to feed the embryo 204. The hull 202 protects the embryo 204 and endosperm 206 during germination, especially from the handling and processing involved in malting. At one end of the grain 200 with the embryo 204, the hull 202 includes a small gap referred to as a micropyle 212, which is where water and other solutes enter the grain 200. As mentioned, the addition of the enzymes during steeping 110 leads to faster opening of the micropyle 212 and, thus, faster absorption of water and solutes in the water.

Steeping 110 is performed until rootlets and acrospires just protrude through the hull 202 of the barley grain 200. The grain 200 at this stage may be referred to as a “chitted malt.” The chitted malt is transported to a germination box for a step of germinating 120 as shown in FIG. 1. According to the presently disclosed method, Gibberellic acid (GA3) and additional β-glucanase (or β-glucanase and xylanase) are added to the grain. In one or more embodiments, the GA3 and β-glucanase are sprayed onto the grain as part of an aqueous solution. In one or more such embodiments, the aqueous solution comprises GA3 in an amount in a range from 0.023 ppm to 1.82 ppm and β-glucanase (or β-glucanase and xylanase) in an amount in a range from 25 ppm to 100 ppm by weight of the chitted grain. While GA3 is preferably applied to the grain during germinating 120, GA3 may additionally or alternatively be added to the water during steeping 110.

Because of the opened micropyle 212, the GA3 and β-glucanase are able to penetrate the grain 200, facilitating faster and more effective water absorption and enzyme activity. The aleurone layer 210 is rapidly stimulated, leading to the secretion of endogenous enzymes sooner than in conventional methods. The endogenous and exogenous enzymes speed the degradation of the cell walls of the endosperm 206. In this way, the germination cycle of the malting process is accelerated. Additionally, enhanced control over the barley modification process is provided as evidenced by the ratio of soluble protein and total protein (S/T) developed during the accelerated process, which was reasonably comparable to the S/T ratio of a conventional, non-accelerated modification process.

During germinating 120, the grain is kept at a temperature in a range from 16° C. to 22° C. Additionally, during germinating 120, humid air is flowed through the germination box. In one or more embodiments, the air has a relative humidity in a range of 95% to 100%. The air may be blown through the germinating grain from the underside to provide sufficient aeration of the grains. Further, the grain may be turned regularly, e.g., using a rotating screw, a rotating drum, or through automated turning systems in a germination box, to ensure uniform moisture and temperature distribution, which is important for consistent grain germination and growth. After completing the step of germinating 120, the grain may be referred to as “green malt.”

From FIGS. 4A-4E, it can be seen that several important actions are taken during steeping 110 and germinating 120 that allow for acceleration of the malting process. From FIGS. 4A and 4B, it can be seen that, the malting process begins with the steeping phase in which the addition of exogenous β-glucanase/xylanase starts the modification of the grain kernels. As shown in FIGS. 4A and 4C, the initial modification during steeping expedites the movement of water into the kernels, leading to a swift opening of the micropyle. This allows the additional exogenous β-glucanase/xylanase and GA3 to penetrate the kernel at the onset of germination, facilitating faster and more effective water absorption and enzyme activity. According to FIGS. 4A and 4D, as GA3-aided water infiltration continues, the aleurone layer is rapidly stimulated, leading to the secretion of endogenous enzymes sooner than in conventional malting processes. These enzymes break down the reserves (endosperm) in the kernel, which is essential for the modification needed in malting. Finally, in FIGS. 4A and 4E, the penetration of external enzymes simultaneously with the early activity of endogenous enzymes results in more effective degradation of the cell walls. This integrated approach significantly reduces germination time and leads to a faster malting process. The combined effect of these refined steps results in a substantial increase in malting efficiency, with a quicker germination cycle and a more controlled modification process.

After obtaining the green malt, the green malt undergoes kilning 130 to reduce the moisture content of the green malt. During kilning 130, the moisture content of the green malt is reduced to about 3.0%-6.0% by weight, more particularly to 3.5%-5.0% by weight. In embodiments, kilning 130 takes place at a temperature in the range of 40° C. to 90° C. for a time of 22 hours to 36 hours, more particularly 24 hours to 30 hours.

After kilning 130, the grain undergoes a cleaning step 140 to remove sprouts and roots, leaving the malt. The malt is then binned, blended, and packaged for shipping.

In addition to the reduced steeping and germination time, the presently disclosed method also provides the surprising and unexpected benefit of reducing the levels of deoxynivalenol (DON) contamination present in the finished malt. DON contamination is caused by secretions from Fusarium fungal infection of the kernel. The Fusarium fungal growth may be primarily localized on the surface of the kernel, but the Fusarium can also penetrate the kernel endosperm, which is harder to mitigate and which compromises the quality of the malt.

During a conventional malting process, the barley grain has a first concentration of DON caused by Fusarium infection. After steeping, the DON concentration typically decreases to a second concentration that may be half or less of the first concentration. However, after germinating and kilning, the Fusarium fungus grows, increasing the concentration of DON to a third concentration in the finished malt that is about as high or higher than the first concentration. For example, Applicant measured the concentration of DON on a batch of malted grain (specifically, barley). The first concentration was 2.50 ppm, and after steeping, the second concentration decreased to about 0.54 ppm. Despite the significant decrease in DON concentration, Fusarium fungal growth during the germinating and kilning steps caused an increase of DON concentration to 2.70 ppm in the finished malt, which is greater than the first concentration. Table 1, below, provides a summary of the DON concentration for various conventional batches of barley grains, including the example batch described above (Batch 1).

TABLE 1
DON Concentration of Conventional Malted Grains

| | Concentration in | Concentration After | Concentration in |
|---|---|---|---|
| | Barley | Steeping | Finished Malt |
| Batch | (ppm) | (ppm) | (ppm) |
| 1 | 2.50 | 0.54 | 2.70 |
| 2 | 2.10 | 0.28 | 2.10 |
| 3 | 2.80 | 1.10 | 7.00 |
| 4 | 11.20 | 4.70 | 10.40 |
| 5 | 1.90 | 0.64 | 1.70 |

By contrast, the malted grain (finished malt) according to the present disclosure has a third concentration of DON that is 70% or less, 60% or less, or 50% or less of the first concentration of DON in the grain. That is, the final DON concentration does not rebound to the level of the first concentration of DON as it does for conventional batches of malt. In one or more embodiments, the third (final) concentration of DON of the malted grain is 2.0 ppm or less, in particular 1.5 ppm or less, and most particularly 1.0 ppm or less.

### EXPERIMENTAL EXAMPLE

Table 2, below, includes the steps of the malting process and the time of each step for a conventional malting process and for the accelerated malting process according to the present disclosure. The steps reflect the time for malting processes utilizing the same equipment, including 3 steeping tanks, 6 germination boxes, and 3 kilns.

TABLE 2
Comparison of Malting Time between Conventional
and Accelerated Process

| | Conventional | Accelerated |
|---|---|---|
| Process Step | Process (Hours) | Process (Hours) |
| Steep Vessel Cleaning | 6 | 6 |
| Barley Wash & Steep In | 3 | 3 |
| Steep (Wet Phase) | 20 | 20 |
| Steep (Dry Phase) | 13 | 4 |
| Germ Vessel Cleaning | 3 | 3 |
| Steep Out/Germination In | 3 | 3 |
| Germination | 78 | 63 |
| Germination Out/Kiln In | 3 | 3 |
| Kiln | 24 | 24 |
| Kiln Out | 3 | 3 |
| Down Time | 0 | 6 |
| Total Time: | 153 | 135 |

As can be seen from Table 2, the conventional process and the accelerated process according to the present disclosure each include the same processing steps. However, the steeping step is shorter in the accelerated process. In particular, the immersion portion of steeping is the same length, but the dry phase time is reduced by 9 hours. Additionally, the germinating step is shorter in the accelerated process by 15 hours. Thus, after the steeping step and the germinating step, the accelerated process is 24 hours faster. Further, the kilning time for the accelerated process is the same, but based on the malting equipment setup, there is some additional down time of about 6 hours. As such, the total time savings of the accelerated process is about 18 hours. Additionally, because of the increased throughput of the accelerated process, an additional 41,714 MT/year of malt is able to be produced using the equipment setup described.

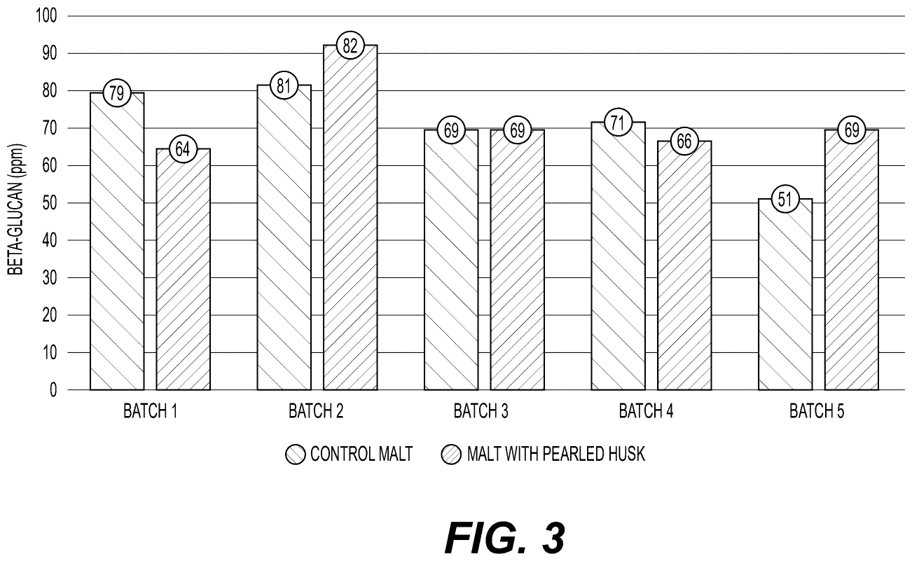

Applicant also found that the malt produced according to the disclosed accelerated malting process was substantially indistinguishable from or improved over malts produced according to the conventional process. In particular, the addition of the exogenous β-glucanase to accelerate the malting process was not evident in the final product, indicating that the enzyme was utilized for modification of the barley. FIG. 3 provides a graph of β-glucan levels in malt from five batches produced according to the disclosed process as compared to the same malt with pearled husk. As can be seen in FIG. 3, the β-glucan levels for the malt produced according to the present disclosure remains substantially the same after pearling. Should the β-glucanase not have been effectively absorbed and utilized within the barley kernels, one might assume lower β-glucan levels in the congress wort as a result of continued degradation of β-glucan by the β-glucanase residing in the husk at the congress mash temperature of 45° C. However, in scenarios where the husk was pearled prior to creating congress wort, a significant increase in beta-glucan levels would have been observed, potentially multiplying, indicating that without successful penetration and utilization of β-glucanase within the barley kernels, the expected outcome would not merely be the maintenance of baseline levels but a substantial elevation. This indicates that the enzymes acted as a process aid and successfully penetrated the malt endosperm in the disclosed accelerated malting process.

In terms of other relevant parameters, Applicant found that the malt produced according to the disclosed accelerated malting process was customizable to a variety of different malt profiles. For batches produced to meet premium distiller standards, the malt exhibited a high diastatic power (DP) of 279 and 313°L (°Lintner), an α-amylase of 120.8 and 122.1 DU (dextrinizing units), a free amino nitrogen (FAN) of 461 and 466 ppm, and a Sol of 10.31% and 10.30%. Further, certain batches exhibited exceptional modification levels, such as friability of 89.3% and 89.9%, viscosities of 1.41 and 1.42 cp, β-glucan levels of 70 and 81 ppm, and S/T ratios of 48.3% and 58.2%. For certain craft brewing specifications, the batches were produced to exhibit a lower DP of 127 and 139°L (°Lintner), β-glucan levels of 71 and 73 ppm, and high friability of 92% and 94.4%.

Additionally, as discussed above, Applicant found that the concentration of DON decreased from the initial concentration of DON on the barley grain when malted according to the presently disclosed method. Table 3, below, provides a comparison of initial DON concentration and final DON concentration for conventional malted grains and grains malted according to the presently disclosed method. In particular, the conventional malting process was a 4-day malting process. Two inventive malting processes were performed, including a 2.5-day malting process and a 3-day malting process.

TABLE 3
Concentration of DON on Malted Grains,
Conventional and Inventive

| | | | Concentration | Concentration |
|---|---|---|---|---|
| | | Concentration | in Finished | in Finished |
| | | in Finished | Malt - | Malt - |
| | Concentration | Malt - | Inventive | Inventive |
| | in Barley | Conventional | (2.5 Day) | (3 Day) |
| Batch | (ppm) | (ppm) | (ppm) | (ppm) |
| 1 | 2.8 | 7.0 | — | 1.7 |
| 2 | 2.5 | 2.7 | 1.1 | 1.4 |
| 3 | 1.8 | 1.7 | 0.9 | 1.0 |

As can be seen from Table 3, the final DON concentration in the finished malt for the conventional method was about as high or higher than the initial DON concentration for each of the batches of barley grain. For both of the inventive malting processes, the final DON concentration in the finished malt was lower. For the first batch, the final DON concentration was reduced by 75.7% as compared to the conventional malting process. For the second batch, the final DON concentration was reduced by 48.1% (3-day) and 59.3% (2.5-day) as compared to the conventional malting process, and for the third batch, the final DON concentration was reduced by 41.2% (3-day) and 47.1% (2.5-day) as compared to the conventional malting process. Thus, the presently disclosed method minimizes fungal growth, thereby decreasing DON contamination.

Accordingly, the disclosed accelerated malting process utilizes a synergistic combination of GA3 and β-glucanase to decrease the steeping and germinating times. In this way, malt production can be significantly increased while utilizing the same equipment or with minimal additional capital expenditure for additional steeping vats, germinating boxes, and kilns.

Furthermore, the accelerated malting process described herein is more sustainable than conventional processes in particular because the disclosed process consumes less electricity. A significant decrease in electricity consumption results from the reduction in germination time. In a specific example of distillers malt production, Applicant observed a 12.1 kW/MT reduction in electricity usage, equating to a 17.2% decrease in consumption across 40 batches, using the disclosed accelerated malting process. In another specific example of brewer's malt production, Applicant observed a 6.6 kW/MT reduction in electricity usage, equating to a 12.1% decrease across 70 batches, using the disclosed accelerated malting process.

All references, including publications, patent applications, and patents cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of accelerating a malting process, the method comprising:
steeping a grain in water comprising β-glucanase to produce chitted malt;
germinating the chitted malt to produce green malt, wherein, during germinating, an aqueous mixture comprising β-glucanase and Gibberellic acid is applied to the chitted malt; and
kilning the green malt to reduce the moisture content to produce a batch of malt.

2. The method of claim 1, wherein the water comprises β-glucanase in an amount in a range from 25 ppm to 100 ppm by weight of the grain.

3. The method of claim 1, wherein the water further comprises xylanase.

4. The method of claim 1, wherein steeping is performed for a time of less than 30 hours.

5. The method of claim 1, wherein steeping comprises a wet phase and a dry phase and wherein the dry phase is performed for a time of 10 hours or less.

6. The method of claim 1, wherein the aqueous mixture further comprises xylanase.

7. The method of claim 1, wherein the aqueous mixture comprises Gibberellic acid in an amount in a range from 0.023 ppm to 1.82 ppm by weight of grain.

8. The method of claim 1, wherein the aqueous mixture comprises β-glucanase in an amount in a range from 25 ppm to 100 ppm by weight of grain.

9. The method of claim 1, wherein the chitted malt is germinated at a temperature in a range from 16° C. to 22° C.

10. The method of claim 1, wherein the chitted malt is germinated at a relative humidity in a range of 95% to 100%.

11. The method of claim 1, wherein the chitted malt comprises a moisture content in a range from 40% to 50% by weight.

12. The method of claim 1, wherein the malt comprises a moisture content in a range from 3.0% by weight to 6.0% by weight.

13. The method of claim 1, wherein kilning is performed at a temperature in the range of 40° C. to 90° C. for a time of 22 hours to 36 hours.

14. The method of claim 1, wherein a first wort prepared from a first portion of the batch of the malt comprises a first β-glucan level, wherein a second wort prepared from a second portion of the batch of malt, after pearling to remove husks, comprises a second β-glucan level, and wherein the second β-glucan level is within 30 ppm of the first β-glucan level.

15. The method of claim 1, wherein, during steeping and germinating, the β-glucanase penetrates the grain to accelerate the steps of steeping and germinating.

16. The method of claim 1, wherein, for a given time period and for a given equipment setup, the method produces more batches of malt than for a method in which β-glucanase is not used during steps of steeping and germinating.

17. The method of claim 1, wherein the grain comprises at least one of barley, wheat, oat, or rye.

18. The method of claim 1, wherein, prior to the steeping, the grain comprises a first concentration of deoxynivalenol (DON), wherein the batch of malt comprises a second concentration of DON, and wherein the second concentration is 70% or less of the first concentration.

19. The method of claim 18, wherein the second concentration of DON is 2.0 ppm or less.

20. A malting process, comprising:
steeping a grain in water, wherein the water comprises an enzyme component selected from a group consisting of β-glucanase, xylanase, and combinations thereof and wherein, during steeping, the enzyme component starts an initial modification of the grain;
germinating the grain while applying water comprising Gibberellic acid and the enzyme component to the grain, wherein the initial modification expedites movement of water into the grain by increasing the rate at which micropyles of the grain open and wherein the Gibberellic acid and the enzyme component penetrate the grain at the onset of germinating to facilitate faster and more effective water absorption and enzymatic activity than applying water alone;
stimulating an aleurone layer of each grain to cause secretion of endogenous enzymes as the water comprising Gibberellic acid and the enzyme component continues to infiltrate the grain, wherein the endogenous enzymes break down reserves in the grain to cause continued modification of the grain; and
degrading of cell walls within the grain through the use of the enzyme component that has infiltrated the grain and the endogenous enzymes such that modification of the grain takes less time than through modification using the endogenous enzymes alone.

21. A batch of malted grain formed through accelerated malting of a grain, the grain having an initial concentration of deoxynivalenol (DON) and the batch of malted grain having a final concentration of DON,
wherein the final concentration of DON is 70% or less of the initial concentration of DON.

22. The batch of malted grain of claim 21, wherein the final concentration of DON is 2.0 ppm or less.

23. The batch of malted grain of claim 21, wherein the grain comprises at least one of barley, wheat, oat, or rye.
